(19) United States
(12) Patent Application Publication
(10) Pub. No.: US 2009/0275035 A1
(43) Pub. Date: Nov. 5, 2009

(54) PRIMERS FOR HUMAN MITOCHONDRIAL HYPERVARIABLE REGION AND METHOD FOR DETECTING HUMAN DNA BY USING THE PRIMERS

(75) Inventor: Man Ning WANG, Sanjhih Township (TW)

Correspondence Address: Muncy, Geissler, Olds & Lowe, PLLC, P.O. BOX 1364, FAIRFAX, VA, 22038-1364, US

(73) Assignee: PREVISION MEDICAL CORPORATION, Taipei (TW)

(21) Appl. No.: 12/398,760

(22) Filed: Mar. 5, 2009

(30) Foreign Application Priority Data
Oct. 12, 2007 (TW) 096138125

(51) Int. Cl. C12Q 1/68; C07H 21/04

(52) U.S. Cl. 435/6; 536/24.33

(57) ABSTRACT

The present invention relates to primer pairs for human mitochondrial hypervariable region and a method for detecting human DNA by using the primer pairs.

### FIELD OF THE INVENTION

The present invention relates to primers for human mitochondrial hypervariable region and method for detecting human DNA by using the primers.

### BACKGROUND OF THE INVENTION

The non-coding control region of mitochondrial DNA (mtDNA) is approximately 1.1 kb in length with frequent variations in the DNA sequence (Chen et al., 2002). The haplotype of the control region is diversified enough for individual identification and the maternal lineage of mtDNA is good for anthropological studies.

The nucleotide variation in the control region is highly concentrated in clusters. Among them, a 342-bp segment (from nucleotides (nts) 16024 to 16365) was first described as hypervariable region I (HVR-I) by Greenberg et al. (Greenberg, B. D., Newbold, J. E., Sugino, A., 1983. Intraspecific nucleotide sequence bariability surrounding the origin of replication in human mitochondrial DNA, Gene 21, 33-49). However, authors of later studies tended to use arbitrarily defined HVR-I segments (Ward et al., 1991; Piercy et al., 1993; Pult et al., 1994; Batista et al., 1995; Graven et al., 1995; Comas et al., 1996; Cõrte-Real et al., 1996; Kolman et al., 1996; Santos et al., 1996; Watson et al., 1996; Lee et al., 1997; Mateu et al., 1997; Baasner et al., 1998; Delghandi et al., 1998; Lutz et al., 1998; Parson et al., 1998; Pfeiffer et al., 1998, 1999, 2001; Rando et al., 1998; Salas et al., 1998; Seo et al., 1998; Krings et al., 1999; Meyer et al., 1999; Orehov et al., 1999; Crespillo et al., 2000; Dimo-Simonin et al., 2000; Green et al., 2000; Helgason et al., 2000; Stoneking, 2000; Yao et al., 2000a, 2000b, 2002a, 2002b; Brakez et al., 2001; Fucharoen et al., 2001; Huoponen et al., 2001; Nasidze and Stoneking, 2001; Qian et al., 2001; Chen et al., 2002; Kivisild et al., 2002; Yao and Zhang, 2002; Fuselli et al., 2003; Brandstätter et al., 2004; Pereira et al., 2004). Such an arbitrary creates a problem when comparing data from different ethnic groups.

For example, the genetic diversity of HVR-I was reported to be 0.985 in both the Japanese (Seo et al., 1998) and Tuareg (Mateu et al., 1997) despite the fact that 404-bp and 360-bp segments, respectively, were referred to as HVR-I when generating these data. Therefore, researchers have to arbitrarily select a segment as HVR-I when comparison of among various ethnic groups is needed. For example, the segment nts 16090-16365 was defined as HVR-I in order to compare 26 European populations (Helgason et al., 2000), whereas the segment nts 16024-16388 was chosen as HVR-I to compare 19 populations (Nasidze and Stoneking, 2001). Therefore, each reference designs a primer for HVR-1 from the arbitrarily defined HVR-1, and thus all the designed primers contain polymorphic sites. Such polymorphic sites vary with ethnic group or individual, i.e. there is no primer suitable for everyone or every ethnic group currently. There is no reason not to use a common HVR-I for the study of genetic diversity.

In view of medical jurisprudence, archaeology, criminal identification, anthropology, and applications which require to identify whether human DNA remains, there needs a method for effectively identifying individual or ethnology.

Accordingly, the present inventor re-defined a hypervariable region by calculating the genetic diversity based on the human mitochondrial DNA retrieved from GenBank and designed and synthesized a primer suitable for detecting human DNA accordingly, and thus completed the present invention.

### SUMMARY OF THE INVENTION

According to the present invention, by calculating the genetic diversity based on the human mitochondrial DNA retrieved from GeneBank, we found that nucleotide sequences (nts) 16126-16362 (the 237-bp) had a global genetic diversity of 0.9905 and nts 16209-16362 (the 154-bp) had a global diversity of 0.9735, and thus named the 237-bp segment the redefined HVR-I (rHVR-I) and named the 154-bp segment the short version of HVR-I (sHVR-I). The present inventor thus designed primers based on the above segments, which can be used for detecting whether human DNA exists and for identifying individual or ethnology.

The present invention provides a primer pair for human mitochondrial hypervariable region, which comprises the following primers (A), (B), and (C):
(A) one primer having the following nucleotide sequence:

| | |
|---|---|
| (a1) 5′-TGGTCAAGGGACCCCTATCT-3′; | (SEQ. NO. 1) |
| or | |
| (a2) 5′-YCCTATCTGAGGGGGGTC-3′; | (SEQ. NO. 2) |

(wherein Y represents C or T)
(B) one primer having the following nucleotide sequence:

| | |
|---|---|
| (b1) 5′-TCGTACATTACTGCCAGYCA-3′; | (SEQ. NO. 3) |
| or | |
| (b2) 5′-CTGCCAGYCACCATGAATAT-3′; | (SEQ. NO. 4) |

(wherein Y represents C or T)
(C) a primer having the following nucleotide sequence:

| | |
|---|---|
| 5′-YCCYCATGCTTACAAGCAAG-3′; | (SEQ. NO. 5) |

(wherein Y represents C or T).

According to the above primer pairs, primers SEQ. NO. 3 and SEQ. NO. 1 together yielded a 309-bp segment within the flanking primer sequences, primers SEQ. NO. 4 and SEQ. NO. 2 together yielded a 287-bp segment within the flanking primer sequences, the two segments are useful for analyzing HVR-1. Moreover, primers SEQ. NO. 5 and SEQ. NO. 1 together yielded a 214-bp segment within the flanking primer sequences, and primers SEQ. NO. 5 and SEQ. NO. 2 together yielded a 202-bp segment within the flanking primer sequences. Therefore, in case of requiring a short DNA segment, the later two primer pairs can be used for analyzing shorter segment in HVR-1.

According to the primer pair, it can be used for detecting human DNA.

The present invention also provides a method for human DNA, which comprises the following steps:

(1) isolating DNA from testing specimen;

(2) subjecting the isolated DNA to polymerase chain reaction in the presence of the primer pair to amplify the nucleotide sequence segment, wherein the primer pairs comprises the following primers (A), (B), and (C):
(A) one primer having the following nucleotide sequence:

| | |
|---|---|
| (a1) 5′-TGGTCAAGGGACCCCTATCT-3′; | (SEQ. NO. 1) |
| or | |
| (a2) 5′-YCCTATCTGAGGGGGGTC-3′; | (SEQ. NO. 2) |

(wherein Y represents C or T)
(B) one primer having the following nucleotide sequence:

| | |
|---|---|
| (b1) 5′-TCGTACATTACTGCCAGYCA-3′; | (SEQ. NO. 3) |
| or | |
| (b2) 5′-CTGCCAGYCACCATGAATAT-3′; | (SEQ. NO. 4) |

(wherein Y represents C or T)
(C) a primer having the following nucleotide sequence:

| | |
|---|---|
| 5′-YCCYCATGCTTACAAGCAAG-3′; | (SEQ. NO. 5) |

(wherein Y represents C or T);
   and

(3) decoding the amplified segment and comparing with the sequence of individual or that stored in GenBank database to detect human mitochondrial hypervariable region and determine the DNA.

### BRIEF DESCRIPTION OF DRAWINGS

FIG. 1 shows the distribution and frequency of polymorphisms in the mitochondrial segment nts 16024-16569. The frequency of nucleotide variation among 1473 individuals retrieved from public databases is expressed in terms of percentage (The top panel). Small boxes (labeled TAS, mt5, and mt3L) indicate the cis-elements, arrows indicate the locations of PCR primers, lines with numbers on both ends (5′ and 3′) represent various HVR-Is reported by different authors.

FIG. 2 shows hypervariable sites in the segment nts 16024-16569. Shorting the segment encompassing HVR-I from the 5′ end downstream (A) or from the 3′ ends upstream (B) identifies 12 and 6 polymorphic sites with Δh>4.5×10⁻⁴ respectively, before reaching a drastic decrease in diversity.

FIG. 3. shows that PCR-producible rHVR-I and sHVR-I can be produced by using highly conserved primers. DNA extracted from 3 different formalin-fixed paraffin-embedded tissues were amplified by using primer pairs SEQ. NO. 3 and SEQ. NO. 1 combined (lane 1, 2, 3, 4), SEQ. NO. 5 and SEQ. NO. 1 combined (lane 5, 6, 7, 8), SEQ. NO. 4 and SEQ. NO. 2 combined (lane 9, 10, 11, 12) or SEQ. NO. 5 and SEQ. NO. 2 combined (13, 14, 15, 16). The PCR products containing rHVR-I (lanes 1-4; lanes 9-12) are 309 bp/287 bp in length, whereas those containing sHVR-I (lanes 5-8; lanes 13-16) are 214 bp/202 bp in length. (M: 100 bp molecular weight marker, arrows indicate the positions for 200 bp and 300 bp bands, lanes 4, 8, 12, 16 were blank control).

### DETAIL DESCRIPTION OF THE INVENTION

### Examples

### 2.1. Data Collection and Correction

In the present invention, we only analyzed DNA sequences that were published in peer-reviewed journals and were available in public databases. Any sequences containing degenerated nucleotides were discarded.

To define the boundaries of HVR-I, the entire mitochondrial genomic sequences were retrieved from GenBank (http://www.ncbi.nlm.nih.gov/) according to the accession numbers provided by the mtDB database. After comparing the sequences from these two databases, those with the same accession number yet failing to correspond with one another were discarded. Sequences from the same geographic area with identical sequences in the coding region (nts 577-16023) were considered as one entry in our dataset.

To compare the HVR-I diversity among various countries, we used only the sequences that had been reported in literatures and had been uploaded to the databases GenBank and HvrBase++. For the present invention, sequences that encompassed nts 16126-16362 (labeled in the text as rHVR-1) were retrieved from GenBank (13 countries) and from HvrBase++ (12 countries). Because the data quality of HvrBase++ is suboptimal (Amason, 2003), all sequences retrieved from HvrBase++ were checked by comparing with the original ones reported in the literatures, and corrections were made accordingly.

### 2.2. Data Analyses

The mtDNA sequences were aligned by ClustalX 1.83 (Thompson et al., 1997) and modified manually. Genetic diversity (heterozygosity), which is the extent of variation in a given population, is determined by the algorithm h=(1−Σχ²)n/(n−1) where n represents sample size and χ is the frequency of each mtDNA (Tajima, 1989). The calculation was performed by Java programs.

### 2.3. DNA Extraction and PCR

DNA was isolated from formalin-fixed paraffin-embedded tumors according to previously described procedures (Tzen et al, 2001).

The primer pairs for the production of rHVR-I were primer JMT (SEQ. NO. 3) and primer MNW (SEQ. NO. 1) or primer JMT′ (SEQ. NO. 4) and primer MNW′ (SEQ. NO. 2). Those for sHVR-1 were primer HJH (SEQ. NO. 5) and primer MNW (SEQ. NO. 1) or primer HJH (SEQ. NO. 5) and primer MNW′ (SEQ. NO. 2). PCR was carried out in a reaction mixture containing 10 mM Tris-HCl (pH 8.3), 50 mM KCl, 2 mM MgCl₂, 0.2 mM deoxynucleotide triphosphate mix, 0.2 μM of each primer, 5 μL cDNA, and 1.25 U AmpliTaq Gold DNA polymerase (Applied Biosystems, Foster City, Calif.). The PCR reaction was performed with the thermal cycler GeneAmp PCR System 9700 (Applied Biosystems, Foster City, Calif.). The reaction protocol was as follows: 1) denaturation at 95° C. for 10 min; 2) amplification for 35 cycles at 94° C. for 30 sec, 58° C. for 40 sec, and 72° C. for 40 sec; and 3) extension at 72° C. for 10 min.

### 3. Results

### 3.1. Refining the 5′ and 3′ Ends of HVR-I

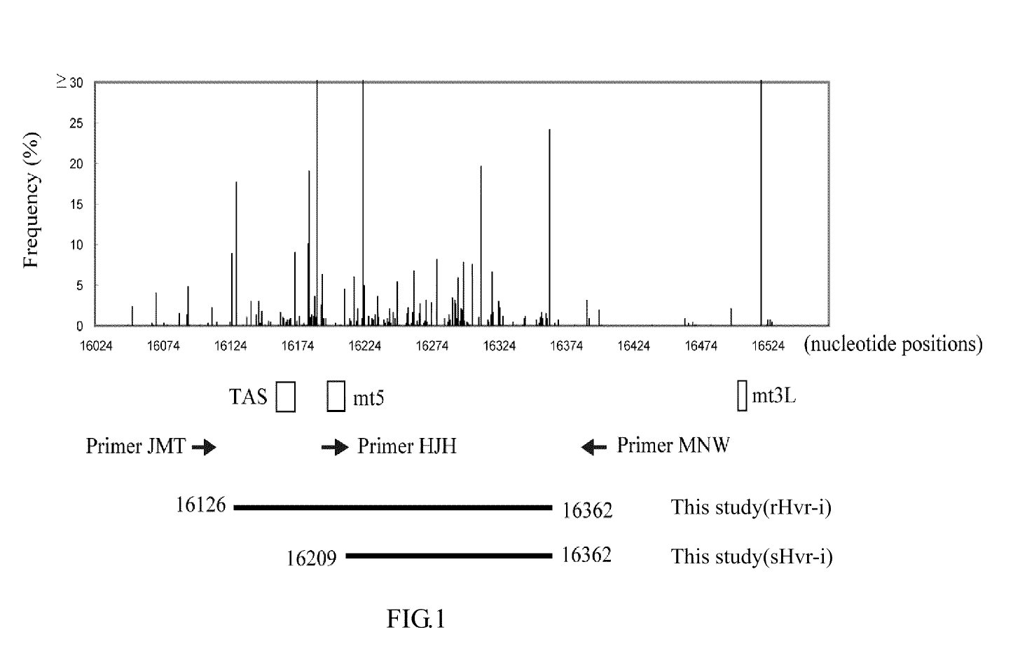

Up to Mar. 1, 2007, a total of 1865 complete human mitochondrial genomic sequences were available from the mtDB database (http://www.genpat.uu.se/mtDB/). These sequences originated from 11 different geographic areas in the world. After selection, a final dataset comprised of 1437 entries was used for further analysis (Table 1). Among them, we found 221 reported polymorphic sites in the segment of nts 16024-16569. The frequencies of these polymorphisms are summarized in FIG. 1.

TABLE 1
Geographic distribution of subjects selected in the present invention

| Region | Subjects | Selected subjects |
|---|---|---|
| Africa | 104 | 96 |
| Asia | 815 | 639 |
| South Asia | 122 | 106 |
| Europe | 682 | 481 |
| America (North) | 6 | 6 |
| America (South) | 3 | 1 |
| Australia | 28 | 24 |
| Melanesia | 50 | 41 |
| Micronesia | 4 | 3 |
| Middle East | 42 | 31 |
| Polynesia | 9 | 9 |
| Total | 1865 | 1437 |

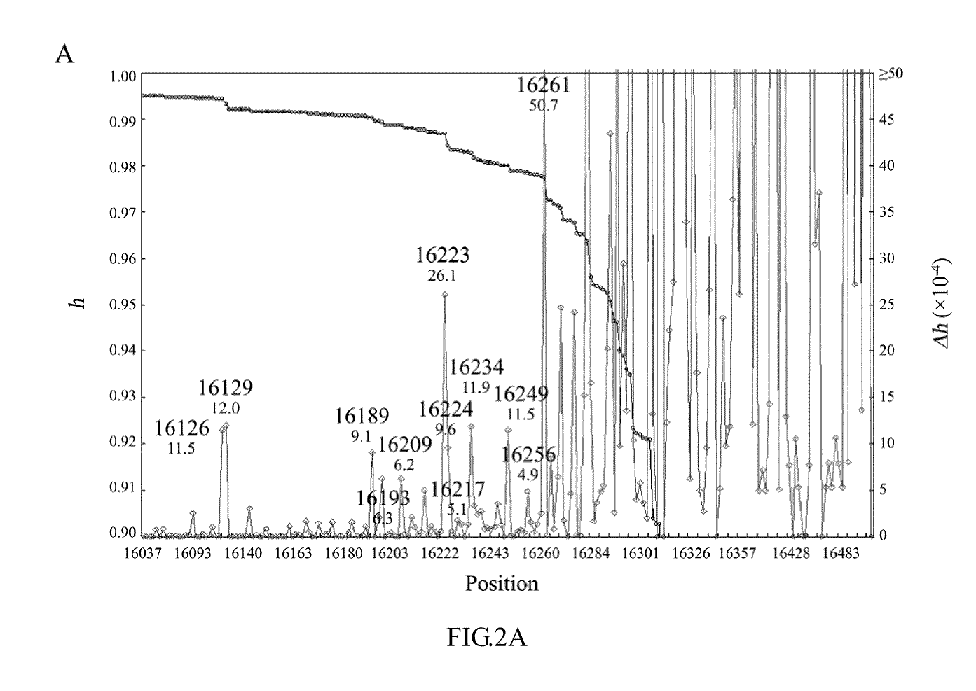

To refine HVR-I, we fixed the 3′ end at nt 16569 and calculated the genetic diversity while moving the 5′ ends downstream, starting from nt 16037 to each polymorphic site (FIG. 2A). The difference (Δh) in genetic diversity between two adjacent polymorphic sites was calculated. The result showed that the average of Δh was 4.5×10⁻³. A tenth of the average value was taken as an arbitrary cutoff point. As shown in FIG. 2A, before reaching a drastic decreases in diversity, 12 polymorphic sites where Δh>4.5×10⁻⁴ were found.

A similar approach was employed to search the potential 3′ ends of the HVR-I. The 5′ end was anchored at nt 16024 and the 3′ ends were moved from nt 16569 upstream to each polymorphic site. The result, presented in FIG. 2B, showed that there were 6 polymorphisms with Δh>4.5×10⁻⁴ before a drastic decrease in diversity occurred. The combination of these 12 possible 5′ ends and 6 possible 3′ ends created 72 versions of HVR-I (Table 2). These potential HVR-Is ranged from 0.6520 to 0.9919 in genetic diversity and 38 bps to 394 bps in length.

TABLE 2
Genetic diversity of all possible versions of HVR-I

| ID # | Region | Length | h | Length/h |
|---|---|---|---|---|
| 1 | 16126-16519 | 394 | 0.9919 | 0.0025 |
| 2 | 16126-16362 | 237 | 0.9905 | 0.0042 |
| 3 | 16129-16519 | 391 | 0.9893 | 0.0025 |
| 4 | 16126-16325 | 200 | 0.9881 | 0.0049 |
| 5 | 16129-16362 | 234 | 0.9873 | 0.0042 |
| 6 | 16126-16311 | 186 | 0.9857 | 0.0053 |
| 7 | 16129-16325 | 197 | 0.9846 | 0.0050 |
| 8 | 16189-16519 | 331 | 0.9845 | 0.0030 |
| 9 | 16126-16304 | 179 | 0.9832 | 0.0055 |
| 10 | 16193-16519 | 327 | 0.9821 | 0.0030 |
| 11 | 16129-16311 | 183 | 0.9819 | 0.0054 |
| 12 | 16209-16519 | 311 | 0.9811 | 0.0032 |
| 13 | 16189-16362 | 174 | 0.9797 | 0.0056 |
| 14 | 16217-16519 | 303 | 0.9795 | 0.0032 |
| 15 | 16129-16304 | 176 | 0.9787 | 0.0056 |
| 16 | 16223-16519 | 297 | 0.9780 | 0.0033 |
| 17 | 16126-16298 | 173 | 0.9779 | 0.0057 |
| 18 | 16193-16362 | 170 | 0.9753 | 0.0057 |
| 19 | 16189-16325 | 137 | 0.9745 | 0.0071 |
| 20 | 16209-16362 | 154 | 0.9735 | 0.0063 |
| 21 | 16224-16519 | 296 | 0.9733 | 0.0033 |
| 22 | 16129-16298 | 170 | 0.9721 | 0.0057 |
| 23 | 16217-16362 | 146 | 0.9708 | 0.0066 |
| 24 | 16234-16519 | 286 | 0.9706 | 0.0034 |
| 25 | 16223-16362 | 140 | 0.9690 | 0.0069 |
| 26 | 16193-16325 | 133 | 0.9683 | 0.0073 |
| 27 | 16189-16311 | 123 | 0.9672 | 0.0079 |
| 28 | 16209-16325 | 117 | 0.9660 | 0.0083 |
| 29 | 16249-16519 | 271 | 0.9653 | 0.0036 |
| 30 | 16256-16519 | 264 | 0.9631 | 0.0036 |
| 31 | 16217-16325 | 109 | 0.9629 | 0.0088 |
| 32 | 16261-16519 | 259 | 0.9608 | 0.0037 |
| 33 | 16223-16325 | 103 | 0.9606 | 0.0093 |
| 34 | 16189-16304 | 116 | 0.9593 | 0.0083 |
| 35 | 16193-16311 | 119 | 0.9572 | 0.0080 |
| 36 | 16209-16311 | 103 | 0.9530 | 0.0093 |
| 37 | 16189-16298 | 110 | 0.9483 | 0.0086 |
| 38 | 16193-16304 | 112 | 0.9472 | 0.0085 |
| 39 | 16217-16311 | 95 | 0.9441 | 0.0099 |
| 40 | 16224-16362 | 139 | 0.9437 | 0.0068 |
| 41 | 16209-16304 | 96 | 0.9423 | 0.0098 |
| 42 | 16234-16362 | 129 | 0.9391 | 0.0073 |
| 43 | 16223-16311 | 89 | 0.9383 | 0.0105 |
| 44 | 16193-16298 | 106 | 0.9338 | 0.0088 |
| 45 | 16217-16304 | 88 | 0.9328 | 0.0106 |
| 46 | 16209-16298 | 90 | 0.9279 | 0.0103 |
| 47 | 16224-16325 | 102 | 0.9263 | 0.0091 |
| 48 | 16249-16362 | 114 | 0.9257 | 0.0081 |
| 49 | 16223-16304 | 82 | 0.9249 | 0.0113 |
| 50 | 16234-16325 | 92 | 0.9213 | 0.0100 |
| 51 | 16256-16362 | 107 | 0.9197 | 0.0086 |
| 52 | 16217-16298 | 82 | 0.9162 | 0.0112 |
| 53 | 16261-16362 | 102 | 0.9141 | 0.0090 |
| 54 | 16223-16298 | 76 | 0.9059 | 0.0119 |
| 55 | 16249-16325 | 77 | 0.9043 | 0.0117 |
| 56 | 16256-16325 | 70 | 0.8942 | 0.0128 |
| 57 | 16261-16325 | 65 | 0.8874 | 0.0137 |
| 58 | 16224-16311 | 88 | 0.8815 | 0.0100 |
| 59 | 16234-16311 | 78 | 0.8551 | 0.0110 |
| 60 | 16224-16304 | 81 | 0.8545 | 0.0105 |
| 61 | 16234-16304 | 71 | 0.8243 | 0.0116 |
| 62 | 16224-16298 | 75 | 0.8178 | 0.0109 |
| 63 | 16249-16311 | 63 | 0.8154 | 0.0129 |
| 64 | 16234-16298 | 65 | 0.7834 | 0.0121 |
| 65 | 16256-16311 | 56 | 0.7831 | 0.0140 |
| 66 | 16249-16304 | 56 | 0.7790 | 0.0139 |
| 67 | 16261-16311 | 51 | 0.7709 | 0.0151 |
| 68 | 16256-16304 | 49 | 0.7330 | 0.0150 |
| 69 | 16249-16298 | 50 | 0.7311 | 0.0146 |
| 70 | 16261-16304 | 44 | 0.7188 | 0.0163 |
| 71 | 16256-16298 | 43 | 0.6800 | 0.0158 |
| 72 | 16261-16298 | 38 | 0.6520 | 0.0172 |

### 3.2. Selection of HVR-I Versions from a Practical Point of View

Although there were a number of HVR-I versions (Table 2), not every of them is flanked by a stretch of conserved DNA sequence suitable for PCR. Scrutinizing the polymorphic sites (FIG. 1) around these potential HVR-I versions, we noticed that PCR primers could be designed on three DNA segments: nts 16094-16113 (primer JMT) (SEQ. NO. 3), nts 16191-16211 (primer HJH) (SEQ. NO. 5), and nts 16402-16383 (primer MNW) (SEQ. NO. 1). The primer JMT had four potential mismatch sites among 1437 individuals: the mismatch rate was 0.14% at nt 16094, 0.21% at nt 16102, 0.35% at nt 16108, and 0.07% at nt 16109. The primer HJH had seven potential mismatch sites: the mismatch rate was 2.51% at nt 16192, 6.33% at nt 16193, 0.97% at nt 16194, 0.90% at nt 16195, 0.28% at nt 16203, 0.21% at nt 16206, and 0.14% at nt 16207. The primer MNW had 5 potential mismatch sites: the mismatch rate was 0.14% at nt 16400, 2.02% at nt 16399, 0.07% at nt 16398, 0.84% at nt 16391, and 3.13% at nt 16390.

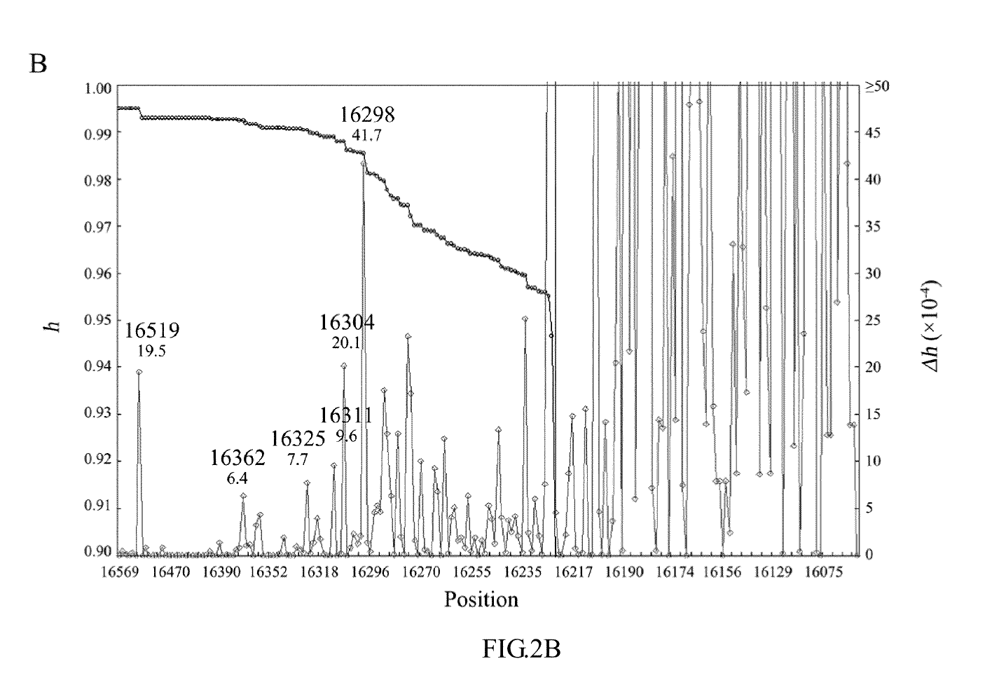

Testing these primer pairs on DNA specimens with poor quality (extracted from paraffin-embedded tissue), we found that the PCR yielded distinct PCR products (FIG. 3). FIG. 3 shows that PCR-producible rHVR-I and sHVR-I can be produced by using highly conserved primers. DNA extracted from 3 different formalin-fixed paraffin-embedded tissues were amplified by using primer pairs SEQ. NO. 3 and SEQ. NO. 1 combined (lane 1, 2, 3), SEQ. NO. 5 and SEQ. NO. 1 combined (lane 5, 6, 7), SEQ. NO. 4 and SEQ. NO. 2 combined (lane 9, 10, 11) or SEQ. NO. 5 and SEQ. NO. 2 combined (13, 14, 15). The PCR products containing rHVR-I (lanes 1-4; lanes 9-12) are 309 bp/287 bp in length, whereas those containing sHVR-I (lanes 5-8; lanes 13-16) are 214 bp/202 bp in length. (M: 100 bp molecular weight marker, arrows indicate the positions for 200 bp and 300 bp bands, lanes 4, 8, 12, 16 were blank control). Primers JMT (SEQ. NO. 3) and MNW (SEQ. NO. 1) together yielded a 309-bp product, primers JMT′ (SEQ. NO. 4) and MNW′ (SEQ. NO. 2) together yielded a 287-bp product, both containing the 237-bp segment (ID #2 in Table 2) within the flanking primer sequences, whereas primers HJH (SEQ. N0.5) and MNW (SEQ. NO. 1) together yielded a 213-bp product, primers HJH (SEQ. NO. 5) and MNW′ (SEQ. NO. 2) together yielded a 202-bp product, both containing the 154-bp segment (ID #20 in Table 2) within the flanking primer sequences. For the sake of simplicity, we named the 237-bp segment (nts 16126-16362) the redefined HVR-I (rHVR-I), and the 154-bp segment (nts 16209-16362) the short version of HVR-I (sHVR-I). As shown in FIG. 1, rHVR-I contains two cis-elements: nts 16157-16172 for the termination-associated sequence (TAS) and nts 16194-16208 for the control element (mt5). In contrast, sHVR-I has no known cis-elements. For the 1437 individuals selected from all over the world at the beginning of the present invention, the global rHVR-I diversity was 0.9905. The global diversity of sHVR-I was 0.9735.

3.3. Genetic diversities of HVR-I A total of 3870 sequences containing rHVR-I were retrieved from public databases. Some sequence errors were found in HvrBase++ and corrections were made accordingly. The major one occurs in the dataset of Germany in that no polymorphisms between nts 16294-16362 were found in 101 sequences of HvrBase++, whereas there were 13 polymorphic sites in 39 sequences of the original report (Lutz, 1998).

Table 3 shows that rHVR-I diversities among these 25 countries ranged from 0.5699 (Panama, Kuna) to 0.9968 (China, Han) and sHVR-I diversities ranged from 0.5699 (Panama, Kuna) to 0.9867 (Mongolia). All ethnic groups with an rHVR-I diversity higher than the global rHVR-I diversity also possess a sHVR-I diversity greater than the global sHVR-I diversity, and vice versa. As shown in Table 3, these ethnic groups were located in Ethipia, Kenya, China, Mongolia, Turkey, and India.

TABLE 3
Genetic diversities of redefined HVR-I and short HVR-I among 25 countries

| | | | h | h | | |
|---|---|---|---|---|---|---|
| Continent | Country | n^{a} | (rHVR-I)^{b} | (sHVR-I)^{c} | Db^{d} | Reference |
| Africa | Algeria | 85 | 0.9420 | 0.9311 | G | Cõrte-Real et al., 1996 |
| | Ethiopia | 270 | 0.9909 | 0.9844 | G | Kivisild et al., 2004 |
| | Kenya(Nairobi) | 100 | 0.9921 | 0.9810 | G | Brandstätter et al., 2004 |
| | Nigeria | 98 | 0.9754 | 0.9611 | G | Watson et al., 1996 |
| | Guinea, São Tomé and Principe^{e} | 95 | 0.9709 | 0.9619 | H | Mateu et al., 1997 |
| | Senegal (Niokolo Mandenka) | 110 | 0.9563 | 0.9461 | H | Graven et al., 1995 |
| | Morocco (Souss) | 50 | 0.9518 | 0.8873 | H | Brakez et al., 2001 |
| Asia | China (Han) | 262 | 0.9968 | 0.9853 | G | Yao et al., 2002a |
| | China (non-Han)^{f} | 295 | 0.9915 | 0.9793 | G | Yao et al., 2002b |
| | China (Tibeto-Burman) | 496 | 0.9934 | 0.9786 | G | Wen et al., 2004 |
| | Mongolia | 103 | 0.9924 | 0.9867 | G | Kolman et al., 1996 |
| | Russia (Kostroma, Ryazan, Kursk) | 103 | 0.9566 | 0.9001 | H | Orekhov et al., 1999 |
| | Yemen | 115 | 0.9794 | 0.9674 | G | Kivisild et al., 2004 |
| | Turkey | 45 | 0.9929 | 0.9768 | G | Comas et al., 1996 |
| | India | 295 | 0.9921 | 0.9767 | G | Kivisild et al., 1999 |
| Oceania | Australia | 54 | 0.9686 | 0.9609 | H | Huoponen et al., 2001 |
| Europe | Iceland | 394 | 0.9655 | 0.9077 | G | Helgason et al., 2000 |
| | Switzerland | 74 | 0.9596 | 0.9145 | G | Pult et al., 1994 |
| | Austria | 100 | 0.9448 | 0.8937 | H | Parson et al., 1998 |
| | France | 50 | 0.9869 | 0.9698 | H | Rousselet and Mangin, |
| | | | | | | 1998 |
| | Germany | 200 | 0.9702 | 0.9320 | H | Lutz et al., 1998 |
| | Norway (Saami) | 61 | 0.7995 | 0.7481 | H | Delghandi et al., 1998 |
| | Spain (Galicia) | 92 | 0.8829 | 0.7387 | H | Salas et al., 1998 |
| America | Canada (Nuu-Chah-Nulth) | 63 | 0.9457 | 0.9211 | G | Ward et al., 1991 |
| | Panama (Kuna) | 63 | 0.5699 | 0.5699 | H | Batista et al., 1995 |
| | Brazil | 92 | 0.9176 | 0.9111 | H | Santos et al., 1996 |
| | Peru | 105 | 0.9564 | 0.8899 | G | Fuselli et al., 2003 |

^{a}simple size.
^{b}rHVR-I: nts 16126-16362
^{c}sHVR-I: nts 16209-16362.
^{d}Database, G: GenBank, H: HvrBase++.
^{e}Adjacent countries.
^{f}Populations included are Lisu, Nu, Sali, Bai, Dai, Zhuang, Tu, and Mongolian.

Descendants of most countries in Africa and Asia had a higher sHVR-I diversity than those from European and American countries (Table 3). More specifically, the h of rHVR-I was 0.9869±0.0133 in Asian countries, 0.9685±0.0193 in African countries, 0.9299±0.0664 in European countries, and 0.8477±0.1857 in American countries. The h of sHVR-I was 0.9689±0.0284 in Asian countries, 0.9504±0.0334 in African countries, 0.8721±0.0911 in European countries, and 0.8230±0.1693 in American countries. This trend is in agreement with the notion that genetic diversity, resulting from mutation accumulation as time passes, roughly reflects the distance on the time scale between the examined population and their common ancestral mtDNA.

### 4. Discussion

### 4.1. Sequence Data Quality Assurance

Sequence errors in mtDNA databases have been noticed in the last few years (Bandelt et al, 2001; Bandelt et al, 2002; Forster, 2003; Dennis, 2003). In this meta-analysis, we kept the quality and accuracy of data by crosschecking DNA sequences between the two publicly accessible databases as well as with the primary data from original sources. By redundant checking, any uncertain data were excluded from our analysis. This approach left us one dataset of 1437 sequences for identifying the HVR-I boundaries and the other dataset of 3870 sequences for comparing the genetic diversity of HVR-I among various ethnic groups. Such an unduly cautious selection limited our data size, but prevented the previous errors from being passed on to the present invention.

### 4.2. Definition of HVR-I: rHVR-I and sHVR-I

In the present invention, the present inventor redefined the 5′ and 3′ ends of HVR-I by analyzing 1437 sequences from individuals around the world. The 237-bp rHVR-I (nts 16126-16362) had a very high genetic diversity (h=0.9905), only second to the 394-bp segment (h=0.9919). They differ 157 bps in length, but only 0.0014 in diversity. The 154-bp sHVR-I (nts 16209-16362) had a high density of diversity (h/length), which ranked third among those with h>0.9700 (Table 2).

Although the diversity of HVR-I has been frequently reported and discussed in literature, it comes to our surprise that all reported HVR-I segments have different 5′ and 3′ ends. As shown in FIG. 1, the 5′ ends ranged from nts 15997 to 16090 and the reported 3′ ends ranged from nts 16362 to 16569. Therefore, the reported HVR-I segments were located within the sequences between nts 15997 and 16569 with an average size of 373±60 bps. They are much longer than the rHVR-I (237 bps), not to mention the sHVR-I (154 bps) (FIG. 1). It should be addressed that one advantage of using a short HVR-I is that it can be employed when specimens are badly degraded. In this regard, it has been reported that the longest amplifiable DNA fragments extracted from 2000 year-old ancient equids from Pompeii remains are between 139 and 360 bps (Di Bemardo et al., 2004).

### 4.3. Comparison Between rHVR-I and sHVR-I

The h of rHVR-I should be greater than or equal to that of sHVR-I because the latter is part of the former. Therefore, it is surprising that the genetic diversities of rHVR-I and sHVR-I in Panama were both low (0.5699) and identical. This can be explained if sHVR-I evolves faster than the other part of rHVR-I. This speculation is consistent with the previous findings, which indicate that most of the fast-evolving sites of rHVR-I are located in sHVR-I. More specifically, there are 32 sites in rHVR-I that were classified as fast-evolving position (Hasegawa et al, 1993; Wakeley, 1993) or had a nucleotide substitution at least twice as fast as the average rate in HVR-I (Meyer et al, 1999). Among them, 23 sites are located in the 154-bp sHVR-I, whereas only 9 sites occur in the remaining region (83-bp in length) of the rHVR-I.

The different evolving rate between rHVR-I and sHVR-I can be explained by the fact that the rHVR-I contains two cis-elements, whereas sHVR-I has no functional domains. Since cis-elements are functional, their mutation will be restricted by the functional requirement. If so, the genetic diversity of rHVR-I is likely to be influenced by adaptation, whereas that of sHVR-I is likely to be a result of neutral evolution. Therefore, we believe the genetic history of a given ethnic group is better evaluated by the h of sHVR-I than the h of rHVR-I. From this standpoint, it is understandable that the difference between European countries and African or Asian countries in sHVR-I is greater than that in rHVR-I.

### 4.4. Conclusion

In the present invention, it defined a 237-bp rHVR-I and a 154-bp sHVR-I in the mitochondrial control region, both of which are polymorphic enough to show differences in genetic diversity among various ethnic groups. The global diversity of rHVR-I is 0.9905 and that of sHVR-I is 0.9735. The diversity of sHVR-I possibly reflects the neutral evolution, whereas the diversity of rHVR-I is additionally influenced by adaptation. Both rHVR-I and sHVR-I are PCR-producible by using highly conserved primers and are short enough to be amplified from badly degraded specimens, allowing a comparison between ethnic groups.

According to the present invention, by using the present primer pairs to subject testing specimen to PCR, it can determine whether human DNA is presence in the testing specimen and can determine its ethnic group based on genetic diversity. Therefore, the primer pairs and the method for detecting human DNA are applicable in medical jurisprudence, archaeology, criminal identification, anthropology, and applications which require to identify whether human DNA remains.

### REFERENCES

1. Ámason, E., 2003. Genetic heterogeneity of Icelanders. Ann. Hum. Genet. 67, 5-16.
2. Baasner, A., Schafer, C., Junge, A., Madea, B., 1998. Polymorphic sites in human mitochondrial DNA control region sequences: population data and maternal inheritance. Forensic Sci. Int. 98, 169-178.
3. Bandelt, H. J., Lahermo, P., Richards, M., Macaulay, V., 2001. Detecting errors in mtDNA data by phylogenetic analysis. Int. J. Legal. Med. 115:64-69.
4. Bandelt, H. J., Quintana-Murci, L., Salas, A., Macaulay, V., 2002. The fingerprint of phantom mutations in mitochondrial DNA data. Am. J. Hum. Genet. 71:1150-1160.
5. Batista, O., Kolman, C. J., Bermingham, E., 1995. Mitochondrial DNA diversity in the Kuna Amerinds of Panama. Hum. Mol. Genet. 4, 921-929.
6. Brakez, Z., Bosch, E., Izaabel, H., Akhayat, O., Comas, D., Bertranpetit, J., Calafell, F., 2001. Human mitochondrial DNA sequence variation in the Moroccan population of the Souss area. Ann. Hum. Biol. 28, 295-307.
7. Brandstätter, A., Peterson, C. T., Irwin, J. A., Mpoke, S., Koech, D. K., Parson, W., Parsons, T. J., 2004. Mitochondrial DNA control region sequences from Nairobi (Kenya): inferring phylogenetic parameters for the establishment of a forensic database. Int. J. Legal Med. 118, 294-306.
8. Chen, M. H., Lee, H. M., Tzen, C. Y., 2002. Polymorphism and heteroplasmy of mitochondrial DNA in the D-loop region in Taiwanese. J. Formos. Med. Assoc. 101, 268-276.
9. Comas, D., Calafell, F., Mateu, E., Perez-Lezaun, A., Bertranpetit, J., 1996. Geographic variation in human mitochondrial DNA control region sequence: the population history of Turkey and its relationship to the European populations. Mol. Biol. Evol. 13, 1067-1077.
10. Cõrte-Real, H. B., Macaulay, V. A., Richards, M. B., Hariti, G., Issad, M. S., Cambon-Thomsen, A., Papiha, S., Bertranpetit, J., Sykes, B. C., 1996. Genetic diversity in the Iberian Peninsula determined from mitochondrial sequence analysis. Ann. Hum. Genet. 60, 331-350.
11. Crespillo, M., Luque, J. A., Paredes, M., Fernandez, R., Ramirez, E., Valverde, J. L., 2000. Mitochondrial DNA sequences for 118 individuals from northeastern Spain. Int. J. Legal Med. 114, 130-132.
12. Delghandi, M., Utsi, E., Krauss, S., 1998. Saami mitochondrial DNA reveals deep maternal lineage clusters. Hum. Hered. 48, 108-114.
13. Dennis C., 2003. Error reports threaten to unravel databases of mitochondrial DNA. Nature. 421:773-774.
14. Di Bernardo, G., Del Gaudio, S., Galderisi, U., Cipollaro, M., 2004. 2000 Year-old ancient equids: an ancient-DNA lesson from pompeii remains. J. Exp. Zoolog. B. Mol. Dev. Evol. 302, 550-556.
15. Dimo-Simonin, N., Grange, F., Taroni, F., Brandt-Casadevall, C., Mangin, P., 2000. Forensic evaluation of mtDNA in a population from south west Switzerland. Int. J. Legal Med. 113, 89-97.
16. Forster P., 2003. To err is human. Ann. Hum. Genet. 67:2-4.
17. Fucharoen, G., Fucharoen, S., Horai, S., 2001. Mitochondrial DNA polymorphisms in Thailand. J. Hum. Genet. 46, 115-125.
18. Fuselli, S., Tarazona-Santos, E., Dupanloup, I., Soto, A., Luiselli, D., Pettener, D., 2003. Mitochondrial DNA diversity in South America and the genetic history of Andean highlanders. Mol. Biol. Evol. 20, 1682-1691.
19. Graven, L., Passarino, G., Semino, O., Boursot, P., Santachiara-Benerecetti, S., Langaney, A., Excoffier, L., 1995. Evolutionary correlation between control region sequence and restriction polymorphisms in the mitochondrial genome of a large Senegalese Mandenka sample. Mol. Biol. Evol. 12, 334-345.
20. Green, L. D., Derr, J. N., Knight, A., 2000. mtDNA affinities of the peoples of North-Central Mexico. Am. J. Hum. Genet. 66, 989-998.
21. Greenberg, B. D., Newbold, J. E., Sugino, A., 1983. Intraspecific nucleotide sequence variability surrounding the origin of replication in human mitochondrial DNA. Gene 21, 33-49.
22. Hasegawa, M., Di Rienzo, A., Kocher, T. D., Wilson, A. C., 1993. Toward a more accurate time scale for the human mitochondrial DNA tree. J. Mol. Evol. 37:347-354.
23. Helgason, A., Sigureth ardottir, S., Gulcher, J. R., Ward, R., Stefansson, K., 2000. mtDNA and the origin of the Icelanders: deciphering signals of recent population history. Am. J. Hum. Genet. 66, 999-1016.
24. Huoponen, K., Schurr, T. G., Chen, Y., Wallace, D. C., 2001. Mitochondrial DNA variation in an aboriginal Australian population: evidence for genetic isolation and regional differentiation. Hum. Immunol. 62, 954-969.
25. Kivisild, T., Bamshad, M. J., Kaldma, K., Metspalu, M., Metspalu, E., Reidla, M., Laos, S., Parik, J., Watkins, W. S., Dixon, M. E., Papiha, S. S., Mastana, S. S., Mir, M. R., Ferak, V., Villems, R., 1999. Deep common ancestry of indian and western-Eurasian mitochondrial DNA lineages. Curr. Biol. 9, 1331-1334.
26. Kivisild, T., Tolk, H. V., Parik, J., Wang, Y., Papiha, S. S., Bandelt, H. J, Villems, R., 2002. The emerging limbs and twigs of the East Asian mtDNA tree. Mol. Biol. Evol. 19, 1737-1751.
27. Kivisild, T., Reidla, M., Metspalu, E., Rosa, A., Brehm, A., Pennarun, E., Parik, J., Geberhiwot, T., Usanga, E., Villems, R., 2004. Ethiopian mitochondrial DNA heritage: tracking gene flow across and around the gate of tears. Am. J. Hum. Genet. 75, 752-770.
28. Kolman, C. J., Sambuughin, N., Bermingham, E., 1996. Mitochondrial DNA analysis of Mongolian populations and implications for the origin of New World founders. Genetics 142, 1321-1334.
29. Krings, M., Salem, A. E., Bauer, K., Geisert, H., Malek, A. K., Chaix, L., Simon, C., Welsby, D., Di Rienzo, A., Utermann, G., Sajantila, A., Paabo, S., Stoneking, M., 1999. mtDNA analysis of Nile River Valley populations: A genetic corridor or a barrier to migration? Am. J. Hum. Genet. 64, 1166-1176.
30. Lee, S. D., Shin, C. H., Kim, K. B., Lee, Y. S., Lee, J. B., 1997. Sequence variation of mitochondrial DNA control region in Koreans. Forensic Sci. Int. 87, 99-116.
31. Lutz, S., Weisser, H. J., Heizmann, J., Pollak, S., 1998. Location and frequency of polymorphic positions in the mtDNA control region of individuals from Germany. Int. J. Legal Med. 111, 67-77.
32. Mateu, E., Comas, D., Calafell, F., Perez-Lezaun, A., Abade, A., Bertranpetit, J., 1997. A tale of two islands: population history and mitochondrial DNA sequence variation of Bioko and Sao Tome, Gulf of Guinea. Ann. Hum. Genet. 61, 507-518.
33. Meyer, S., Weiss, G., von Haeseler, A., 1999. Pattern of nucleotide substitution and rate heterogeneity in the hypervariable regions I and II of human mtDNA. Genetics 152, 1103-1110.
34. Nasidze, I., Stoneking, M., 2001. Mitochondrial DNA variation and language replacements in the Caucasus. Proc. Biol. Sci. 268, 1197-1206.
35. Orekhov, V., Poltoraus, A., Zhivotovsky, L. A., Spitsyn, V., Ivanov, P., Yankovsky, N., 1999. Mitochondrial DNA sequence diversity in Russians. FEBS Lett. 445, 197-201.
36. Parson, W., Parsons, T. J., Scheithauer, R., Holland, M. M., 1998. Population data for 101 Austrian Caucasian mitochondrial DNA d-loop sequences: application of mtDNA sequence analysis to a forensic case. Int. J. Legal Med. 111, 124-132.
37. Pfeiffer, H., Steighner, R., Fisher, R., Mornstad, H., Yoon, C. L., Holland, M. M., 1998. Mitochondrial DNA extraction and typing from isolated dentin-experimental evaluation in a Korean population. Int. J. Legal Med. 111, 309-313.
38. Pfeiffer, H., Brinkmann, B., Huhne, J., Rolf, B., Morris, A. A., Steighner, R., Holland, M. M., Forster, P., 1999. Expanding the forensic German mitochondrial DNA control region database: genetic diversity as a function of sample size and microgeography. Int. J. Legal Med. 112, 291-298.
39. Pfeiffer, H., Forster, P., Ortmann, C., Brinkmann, B., 2001. The results of an mtDNA study of 1,200 inhabitants of a German village in comparison to other Caucasian databases and its relevance for forensic casework. Int. J. Legal Med. 114, 169-172.
40. Pereira, L., Cunha, C., Amorim, A., 2004. Predicting sampling saturation of mtDNA haplotypes: an application to an enlarged Portuguese database. Int. J. Legal Med. 118, 132-136.
41. Piercy, R., Sullivan, K. M., Benson, N., Gill, P., 1993. The application of mitochondrial DNA typing to the study of white Caucasian genetic identification. Int. J. Legal Med. 106, 85-90.
42. Pult, I., Sajantila, A., Simanainen, J., Georgiev, O., Schaffner, W., Paabo, S., 1994. Mitochondrial DNA sequences from Switzerland reveal striking homogeneity of European populations. Biol. Chem. Hoppe. Seyler. 375, 837-840.
43. Qian, Y. P., Chu, Z. T., Dai, Q., Wei, C. D., Chu, J. Y., Tajima, A., Horai, S., 2001. Mitochondrial DNA polymorphisms in Yunnan nationalities in China. J. Hum. Genet. 46, 211-220.
44. Rando, J. C., Pinto, F., Gonzalez, A. M., Hernandez, M., Larruga, J. M., Cabrera, V. M., Bandelt, H. J., 1998. Mitochondrial DNA analysis of northwest African populations reveals genetic exchanges with European, near-eastern, and sub-Saharan populations. Ann. Hum. Genet. 62, 531-550.
45. Rousselet, F., Mangin, P., 1998. Mitochondrial DNA polymorphisms: a study of 50 French Caucasian individuals and application to forensic casework. Int. J. Legal Med. 111, 292-298.
46. Salas, A., Comas, D., Lareu, M. V., Bertranpetit, J., Carracedo, A., 1998. mtDNA analysis of the Galician population: a genetic edge of European variation. Eur. J. Hum. Genet. 6, 365-375.
47. Santos, S. E., Ribeiro-Dos-Santos, A. K., Meyer, D., Zago, M. A., 1996. Multiple founder haplotypes of mitochondrial DNA in Amerindians revealed by RFLP and sequencing. Ann. Hum. Genet. 60, 305-319.
48. Seo, Y., Stradmann-Bellinghausen, B., Rittner, C., Takahama, K., Schneider, P. M., 1998. Sequence polymorphism of mitochondrial DNA control region in Japanese. Forensic Sci. Int. 97, 155-164.
49. Stoneking, M., 2000. Hypervariable sites in the mtDNA control region are mutational hotspots. Am. J. Hum. Genet. 67, 1029-1032.
50. Tajima, F., 1989. Statistical method for testing the neutral mutation hypothesis by DNA polymorphism. Genetics 123, 585-595.
51. Thompson, J. D., Gibson, T. J., Plewniak, F., Jeanmougin, F., Higgins, D. G., 1997. The ClustalX windows interface: flexible strategies for multiple sequence alignment aided by quality analysis tools. Nucleic Acids Res. 25, 4876-4882.
52. Tzen, C. Y., Wu, T. Y., Liu, H. F. 2001. Sequence polymorphism in the coding region of mitochondrial genome encompassing position 8389-8865. Forensic Sci. Int. 120:204-209.
53. Wakeley J., 1993. Substitution rate variation among sites in hypervariable region 1 of human mitochondrial DNA. J. Mol. Evol. 37, 613-623.
54. Ward, R. H., Frazier, B. L., Dew-Jager, K., Paabo, S., 1991. Extensive mitochondrial diversity within a single Amerindian tribe. Proc. Natl. Acad. Sci. U.S.A. 88, 8720-8724.
55. Watson, E., Bauer, K., Aman, R., Weiss, G., von Haeseler, A., Paabo, S., 1996. mtDNA sequence diversity in Africa. Am. J. Hum. Genet. 59, 437-444.
56. Wen, B., Xie, X., Gao, S., Li, H., Shi, H., Song, X., Qian, T., Xiao, C., Jin, J., Su, B., Lu, D., Chakraborty, R., Jin, L., 2004. Analyses of genetic structure of Tibeto-Burman populations reveals sex-biased admixture in southern Tibeto-Burmans. Am. J. Hum. Genet. 74, 856-865.
57. Yao, Y. G., Lü, X. M., Luo, H. R., Li, W. H., Zhang, Y. P., 2000a. Gene admixture in the silk road region of China: evidence from mtDNA and melanocortin 1 receptor polymorphism. Genes Genet. Syst. 75, 173-178.
58. Yao, Y. G., Watkins, W. S., Zhang, Y. P., 2000b. Evolutionary history of the mtDNA 9-bp deletion in Chinese populations and its relevance to the peopling of east and southeast Asia. Hum. Genet. 107, 504-512.
59. Yao, Y. G., Kong, Q. P., Bandelt, H. J., Kivisild, T., Zhang, Y. P., 2002a. Phylogeographic differentiation of mitochondrial DNA in Han Chinese. Am. J. Hum. Genet. 70, 635-651.
60. Yao, Y. G., Nie, L., Harpending, H., Fu, Y. X., Yuan, Z. G., Zhang, Y. P., 2002b. Genetic relationship of Chinese ethnic populations revealed by mtDNA sequence diversity. Am. J. Phys. Anthropol. 118, 63-76.
61. Yao, Y. G., Zhang, Y. P., 2002. Phylogeographic analysis of mtDNA variation in four ethnic populations from Yunnan Province: new data and a reappraisal. J. Hum. Genet. 47, 311-318.

What is claimed is:

1. A primer pair for human mitochondrial hypervariable region, which comprises the following primers (A), (B), and (C):
(A) one primer having the following nucleotide sequence:

| | |
|---|---|
| (a1) 5′-TGGTCAAGGGACCCCTATCT-3′; | (SEQ. NO. 1) |
| or | |
| (a2) 5′-YCCTATCTGAGGGGGGTC-3′; | (SEQ. NO. 2) |

(wherein Y represents C or T)
(B) one primer having the following nucleotide sequence:

| | |
|---|---|
| (b1) 5′-TCGTACATTACTGCCAGYCA-3′; | (SEQ. NO. 3) |
| or | |
| (b2) 5′-CTGCCAGYCACCATGAATAT-3′; | (SEQ. NO. 4) |

(wherein Y represents C or T)
(C) a primer having the following nucleotide sequence:

| | |
|---|---|
| 5′-YCCYCATGCTTACAAGCAAG-3′ | (SEQ. NO. 5) |

(wherein Y represents C or T).

2. The primer pair according to claim 1, which is used for detecting human DNA.

3. A method for human DNA, which comprises the following steps:
(1) isolating DNA from testing specimen;
(2) subjecting the isolated DNA to polymerase chain reaction in the presence of the primer pair according to claim 1 to amplify the nucleotide sequence segment; and
(3) decoding the amplified segment and comparing with the sequence of individual or that stored in GenBank database to detect human mitochondrial hypervariable region and determine the DNA.

4. The method according to claim 3, wherein the polymerase chain reaction is performed once or several times.

5. The method according to claim 3, wherein the testing specimen is blood or tissue.


SEQUENCE LISTING (5 sequences)

SEQ ID NO: 1 - DNA, 20 bp, Artificial Sequence
  Synthetic primer
tggtcaagggacccctatct

SEQ ID NO: 2 - DNA, 18 bp, Artificial Sequence
  Synthetic primer
ycctatctgaggggggtc

SEQ ID NO: 3 - DNA, 20 bp, Artificial Sequence
  Synthetic primer
tcgtacattactgccagyca

SEQ ID NO: 4 - DNA, 20 bp, Artificial Sequence
  Synthetic primer
ctgccagycaccatgaatat

SEQ ID NO: 5 - DNA, 20 bp, Artificial Sequence
  Synthetic primer
yccycatgcttacaagcaag